(19) United States
(12) Patent Application Publication
(10) Pub. No.: US 2017/0135777 A1
(43) Pub. Date: May 18, 2017

(54) SURGICAL ISOLATION DRAPE

(71) Applicant: Eric Ray Behrman, Beavercreek, OH (US)

(72) Inventor: Eric Ray Behrman, Beavercreek, OH (US)

(21) Appl. No.: 15/349,586

(22) Filed: Nov. 11, 2016

Related U.S. Application Data
(60) Provisional application No. 62/254,209, filed on Nov. 12, 2015.

(51) Int. Cl. A61B 46/27; B65D 33/28; A61B 46/00

(52) U.S. Cl. CPC A61B 46/27; A61B 46/40; B65D 33/28

(57) ABSTRACT

A surgical isolation drape comprises a medical-grade flexible container body including an inside surface, a first end that is normally open, thus defining a first mouth, and a second end opposite the first end that is normally open, thus defining a second mouth. The surgical isolation drape also includes a first closure mechanism to close the first mouth. For instance, the first closure mechanism can include a first channel inside of the container body proximate the first mouth. A first closure line passes through the first channel, such that access to the first closure line is provided from a position inside the container body. The surgical isolation drape still further includes a first locking mechanism that engages the first closure line, such that access to the first locking mechanism is provided from a position inside the body.

### CROSS REFERENCE TO RELATED APPLICATIONS

This application claims the benefit of U.S. Provisional Patent Application Ser. No. 62/254,209, filed Nov. 12, 2015, entitled SURGICAL ISOLATION DRAPE, the disclosure of which is hereby incorporated herein by reference.

### BACKGROUND

The present disclosure relates in general to surgical devices, and in particular, to a surgical drape for medical procedures, which provides an isolation area within a surgical site, such as a body cavity.

Minimally invasive surgical techniques provide numerous benefits to a patient undergoing a surgical procedure, including shortened recovery time, reduced chance of side effects, and reduced scarring and other outwardly visible signs of the procedure. By way of example, laparoscopic techniques are often utilized to perform minimally invasive inspection, surgery, and other procedures by gaining access to a body site, such as the abdominal cavity or pelvic cavity, either through one or more small incisions, or via a natural body passage. In this technique, surgical tools are passed through the body to the operative site, typically using openings that are 1 centimeter or less.

### BRIEF SUMMARY

According to aspects of the present disclosure, a surgical isolation drape is provided. The surgical isolation drape comprises a medical-grade flexible container body, and a first closure mechanism. The flexible container body has an inside surface, a first end that is normally open, thus defining a first mouth, and a second end opposite the first end that is normally open, thus defining a second mouth. In this manner, the container body defines a sleeve when the first mouth and the second mouth are each open. The first closure mechanism is attached to the container body, and is operable from a position inside the container body to open and close the first mouth. More particularly, the first closure mechanism is operated to restrict the maximum diameter of the first mouth so as to sealingly close around a tissue of interest and create an isolated surgical environment. In this regard, in some embodiments, the first mouth can also be entirely sealed off, defining a bag.

According to further aspects of the present disclosure, a surgical isolation drape is provided. The surgical isolation drape comprises a medical-grade flexible container body, and a first closure line. The flexible container body includes an inside surface, a first end that is normally open defining a first mouth, and a second end opposite the first end that is normally open defining a second mouth. Moreover, the container body defines a sleeve when the first mouth and the second mouth are each open. The surgical isolation drape also includes a first channel inside of the container body proximate the first mouth. The first closure line passes through the first channel, such that access to the first closure line is provided from a position inside the container body. The surgical isolation drape still further includes a first locking mechanism that engages the first closure line, where access to the first locking mechanism is provided from a position inside the body. In this manner, the first locking mechanism is operable from within the container body.

In operation, the first locking mechanism is transitioned to a locked state to cause the first closure line to restrict a maximum diameter of the first mouth. Yet further, the first closure line can be pulled from within the container body when the first locking mechanism is in an unlocked state to restrict the maximum diameter of the first mouth, e.g., so as to sealingly close around tissue and create an isolated surgical environment. In this regard, in some embodiments, the first mouth can be entirely sealed off, defining a bag, e.g., with a single opening opposite the sealed off first mouth, so long as the first mouth remains in its fully closed off position.

According to yet further aspects of the present disclosure, a method of using a surgical isolation drape is provided. The method comprises inserting, during a surgical procedure, a surgical isolation drape into a cavity of a patient. The surgical isolation drape has features and characteristics as set out above. For instance, the surgical isolation drape comprises a medical-grade flexible container body, and a first closure mechanism. The flexible container body has an inside surface, a first end that is normally open, thus defining a first mouth, and a second end opposite the first end that is normally open, thus defining a second mouth. Here, the container body defines a sleeve when the first mouth and the second mouth are each open. The first closure mechanism is attached to the container body and is operable from a position inside the container body to open and close the first mouth. Moreover, the first closure mechanism is operated to restrict the maximum diameter of the first mouth so as to sealingly close around a tissue of interest and create an isolated surgical environment.

The method also comprises slipping the first mouth of the surgical drape over tissue that is subject to a surgical procedure. The method still further comprises operating the first closure mechanism from within the container body of the surgical drape to seal the container body about the tissue. Yet further, the method comprises locking the first closure mechanism to seal the first mouth, e.g., about the tissue so that a surgical site is contained within the container body, or to entirely seal off the first mouth defining a bag structure.

### BRIEF DESCRIPTION OF THE SEVERAL VIEWS OF THE DRAWINGS

FIG. 1 is a view of a surgical isolation drape according to aspects of the present disclosure herein;

FIG. 2 is a top viewof a surgical isolation drape, according to aspects of the present disclosure;

FIG. 3 is a top viewof a surgical isolation drape, according to further aspects of the present disclosure;

FIG. 4 is a flow chart illustrating a method of performing a surgical procedure using a surgical isolation drape according to aspects of the present disclosure herein;

FIG. 5 is a schematic top view of the surgical isolation drape of FIG. 1 in use during a surgical procedure; and

FIG. 6 is a schematic side view of the surgical isolation drape of FIG. 5.

### DETAILED DESCRIPTION

According to various aspects of the present disclosure, a surgical isolation drape is provided, which is suitable for minimially invasive surgical procedures, including laproscopic, endoscopic, and telesurgical procedures that may require morcellation, tissue excision and removal, surgical repair, other procedures, combinations of the above, etc.

Example Surgical Isolation Drape

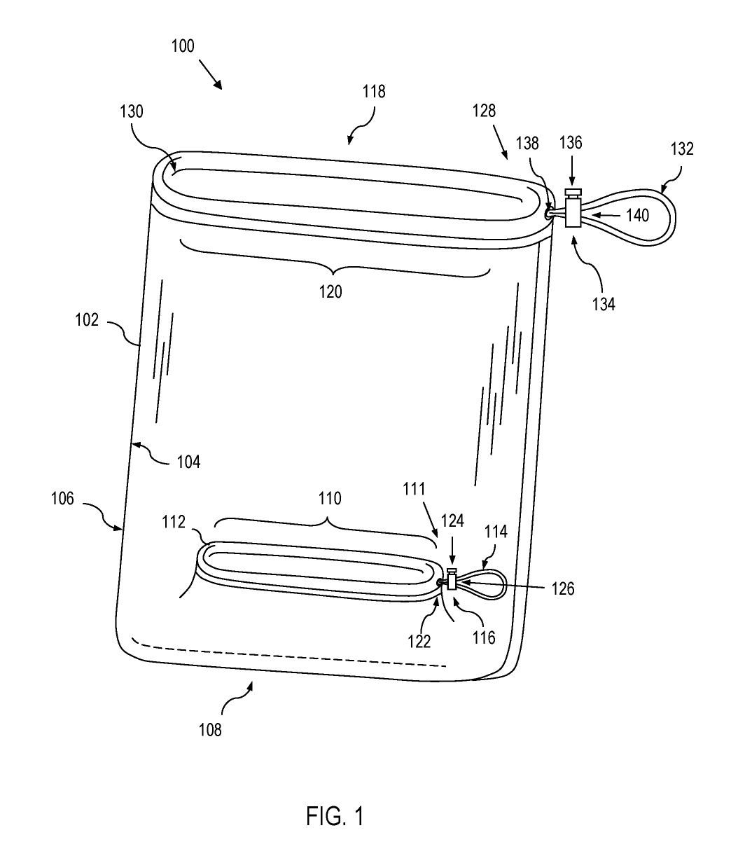

Referring now to the drawings, and in particular, to FIG. 1, a surgical isolation drape 100 is illustrated. The surgical isolation drape 100 includes in general, a medical-grade flexible container body 102 having an inside surface 104 and an outside surface 106. In practice, the container body 102 can be any suitable material. However, in certain embodiments, the flexible container body 102 is a clear material, an elastic material, or a combination thereof. In an example implementation, the container body 102 is a clear, stretchable (e.g., elastic), surgical grade material. The elastic material allows the container body 102 to withstand a typical anticipated use where implements are inserted into the surgical isolation drape to perform the necessary procedure. That is, in this example configuration, the container body 102 is able to withstand modest amounts of stretching, poking, and repositioning of typical surgical instruments without compromising the integrity of the isolation formed by use of the surgical isolation drape.

In some embodiments, the container body 102 may comprise one or more physical layers, e.g., a puncture-resistant, stretchable inner layer, and a moisture proof outer layer. In other embodiments, more than two layers are assembled together. In yet other embodiments, a single layer of material is utilized. In practical embodiments, at least one layer of material of the container body should be nonpermeable to bodily matter including bodily fluids. In yet further embodiments, the container body 102 is nonpermeable to a gas used to inflate the container body 102, as will be described in greater detail herein. By way of example, the container body 102 may comprise polyurethane, latex, nylon flexible fabric, a biocompatible material, combination of the above, etc., that is/are configured to meet sterility and/or other requirements for internal surgery.

As illustrated towards the bottom of the view in FIG. 1, the surgical isolation drape 100 has a first end 108 that is normally open, thus defining a first mouth 110. The surgical isolation drape 100 also includes a first closure mechanism 111. For instance, in the illustrated example of a first closure mechanism 111, a first channel 112 is provided inside of the container body 102 proximate the first mouth 110. A first closure line 114 passes through the channel 112, and exits into the container body 102 such that access to the first closure line 114 is provided from a position inside the container body 102. Also, an optional first locking mechanism 116 engages the first closure line 114 such that access to the first locking mechanism 116 is provided from a position inside the container body 102.

Although the first closure mechanism 111 is illustrated as a first closure line 114 and optionally, a separate first locking mechanism 116, in practice other structures may be utilized to provide a closure function. Also, in some embodiments, a locking function may be integrated into a closure structure, e.g., as in a zip tie or zip lock that integrates both a first closure line 114 and a locking mechanism 116 into a common structure. As a few non-limiting alternative examples, the closure mechanism can include a zipper, a reusable and re-sealable zipper, a zip-close seal structure, a waterproof Velcro or other waterproof fastener, combination thereof, etc.

Moreover, in some embodiments, the first mouth 110 couples to the container body 102 via suitable interconnecting structures, e.g., a shoulder, necked down area, a tapered end, an inward folded structure, etc., as may be necessary to position the first closure mechanism 111 for operation from within the container body 102. An example of a necked area is shown in FIG. 1 as a non-limiting example.

As illustrated towards the top of the view in FIG. 1, the surgical isolation drape 100 also includes a second end 118 opposite the first end 108. The second end 118 is normally open, thus defining a second mouth 120. Accordingly, the container body 102 defines a sleeve when the first mouth 110 and the second mouth 120 are each open. The container body 102 defines a bag when the second mouth 120 is open and the first mouth 110 is sealed closed.

Use of the Surgical Isolation Drape

In use, the first closure mechanism 111, which is attached to the container body 102, is operable from a position inside the container body 102 to open and close the first mouth 110, where the first closure mechanism 111 is operated to restrict the maximum diameter of the first mouth 110 so as to sealingly close around a tissue of interest and create an isolated surgical environment (or to entirely seal closed, the first mouth 110 of the container body 102).

By way of the example configuration of FIG. 1, the first locking mechanism 116 (of the first closure mechanism 111) is operable from within the container body 102. More particularly, a user can transition the first locking mechanism 116 to a locked state (e.g., using a surgical instrument inserted into the container body 102) to cause the first closure line 114 to restrict a maximum diameter of the first mouth 110. Likewise, a user can pull the first closure line 114 (e.g., using a surgical instrument inserted into the container body 102) from within the container body 102 when the first locking mechanism 116 is in an unlocked state to restrict the maximum diameter of the first mouth 110 (or to release restriction of the first mouth 110, where desired). In this manner, the first mouth 110 can be pulled so as to sealingly close around tissue of interest and create an isolated surgical environment. Moreover, the first mouth 110 can be completely closed off, thus creating a surgical isolation bag. This is particularly useful when excising tissue. For instance, the surgical isolation drape 100 can be drawn about tissue to be excised, by sealingly tightening the first mouth 110 thereabout (e.g., about the tissue to be excised). As the tissue is removed via excision, the first mouth 110 can be pulled in (i.e., closed off), thus constricting around the tissue as the tissue is being excised. Once the tissue is fully excised, the first mouth 110 can be completely sealed off, thus containing the excised tissue from other various tissues and organs of a corresponding body cavity, thus forming essentially a contained bag. In this manner, the first mouth 110 is sealed in such a way as to be nonpermeable to bodily matter, including bodily fluids.

In this example implementation, the container body 102 is sufficiently elastic to allow surgical instruments to be inserted within the container body 102 without compromising isolation provided by the surgical isolation drape 100 within a body cavity while instruments are arranged in a standard recognized orientation for a given surgery.

In another example implementation, the first closure line 114 enables the first mouth 110 to be drawn into such sealing relationship with tissue of interest (or to be closed off entirely thus defining a closed end of the container body 102). In this example implementation, the container body 102 of an isolation drape forms a vessel configured to be fillable with a gas defining an inflated drape that expands a body cavity allowing for visualization and access to various tissues and organs of the body cavity. Inflation of the container body 102 is useful to create room inside the container body 102 to insert and manipulate surgical tools. Moreover, inflation of the container body 102 can be used to push organs and other body parts near the operative tissue out of the way of the surgical field. Thus, inflation of the container body 102 is useful for minimizing the body tissue that must be touched/manuevered by the surgeon that might otherwise prevent ready access to the operative tissue. For instance, in an example implementation, the isolation drape forms a stretchable, transparent vessel configured to be fillable with a gas allowing the inflated drape to expand the body cavity allowing for visualization and access to various tissues and organs of the body cavity that are of interest for the surgical procedure.

Drawstring Closure Mechanism

In an example implementation, the first channel 112 is implemented as an internal casing positioned on the inside surface 104 of the container body 102. The internal casing has a slit 122 or other suitable opening thus forming a drawstring-type closure mechanism. For instance, in the illustrated example, the first closure line 114 comprises a string that passes through the internal casing and exits the container body 102 about the slit 122 therein so as to exit the casing on the inside the container body 102.

In this example implementation, the first locking mechanism 116 comprises a lock having a release 124 and a string channel 126. The ends of the first closure line 114 pass through the string channel 126, e.g., such that a loop of the first closure line 114 extends from the string channel 126. In an example implementation, the first locking mechanism 116 utilizes a spring-biased member that clamps the first closure line 114 when in the locked state. As an illustrative example, the locking mechanism 116 can include a ball style, barrel style, block style, etc., cord lock. Alternative configurations can include zipper pulls, slip knots, etc.

To draw the first closure line 114 closed, e.g., to close off the first mouth 110, or to pull the first closure line 114 loose to open up the first mouth 110, the first locking mechanism 116 is transitioned to the unlocked state by pressing against the release 124, thus actuating the spring-biased member to temporarily release the clamping action for as long as the spring-bias is compressed by the release 124.

In practice, the first locking mechanism 116 can take any form that can be operated by surgical instruments that are inserted into the container body 102. For instance, in an example implementation, the spring-biased member is coupled to the release 124 so as to allow the locking action of the first locking mechanism 116 to transition between a locked state wherein the first closure line 114 is restricted from sliding through the string channel 126 (release 124 not pressed), and an unlocked state wherein the first closure line 114 can slide through the string channel 126 (release 124 pressed).

Thus, in the illustrative example, the release 124 is implemented as a spring-biased button. Pressing the spring-biased button causes the locking mechanism 116 to transition to the unlocked state. While the release 124 is held pressed, the first closure line 114 can be adjusted, e.g., pulled to close the first mouth 110, or allowed to slacken, thus widening the first mouth 110. After making the necessary adjustments to the first closure line 114, the spring bias of the release 124 can be let go, thus locking the first closure line 114, and hence, restricting the first mouth 110 from widening. The spring-bias of the button corresonding to the release 124 thus automatically transitions the locking mechanism 116 back to the locked state.

In some embodiments, the first closure mechanism can include more than one device that cooperate to allow the user to seal the first end of the container body 102 closed. For instance, the closure line 114 can comprise parallel run strings spaced apart from one another so that pulling the first mouth 110 closed forms a seal via the material of the container body 102 in between the parallel strings closing off the first mouth 110. As another example, a waterproof fastener can be included as a secondary seal that is available when the user pulls the first closure line 114 so as to entirely close off the first mouth 110. For instance, the waterproof fastener can fold over the first mouth 110 thus forming a seal over the drawn in material of the first mouth 110. In some embodiments, the material itself can form a seal. Here, when the first mouth 110 is pulled closed, the material sufficiently closes around itself to form a seal. In yet other embodiments, such as where a zip-seal is used, the mouth is not drawn inward, but rather closed off by working from one end of the mouth 110 towards the opposite end, pulling the zip across as the first mouth 110 is closed off. When the zip is fully drawn, the first mouth 110 is fully closed off, essentially creating a closed-end bag.

Optional Second Closure Mechanism

In some example applications, the second mouth 120 does not include a second closure mechanism 128 opposite the first closure mechanism. In other example applications, the second mouth 120 includes its own closure mechanism 121. In configurations including a second closure mechanism 128 about the second mouth 120, the second closure mechanism 128 can be implemented using any one or more of the structures described above with regard to the first closure mechanism 111. In this regard, the second closure mechanism 128 can be the same as, or different from the first closure mechanism 111. For instance, the first closure mechanism 111 can be a zip lock mechanism and the second closure mechanism 128 can be a drawstring, etc. Alternatively, both the first closure mechanism 111 and the second closure mechanism 128 can be the same structure, e.g., a drawstring structure.

For instance, as an example of a second closure mechanism 128 in the illustrated implementation, the surgical isolation drape 100 also includes a second channel 130 proximate to the second mouth 120. In a manner analogous to that described above with reference to the first closure mechanism 111, a second closure line 132 passes through the second channel 130. Also, a second locking mechanism 134 engages the second closure line 132. The second closure line 132 and the second locking mechanism 134 are analogous to the first closure line 114 and first locking mechanism 116, respectively. In the illustrative example, the second locking mechanism 134 is transitioned to a locked state to cause the second closure line 132 to restrict a maximum diameter of the second mouth 120. Likewise, the second closure line 132 can be pulled when the second locking mechanism 134 is in an unlocked state to restrict the maximum diameter of the second mouth 120. Also analogous to the first closure mechanism 111, the second closure line 132 can be adjusted when the second locking mechanism 134 is in an unlocked state to open/enlarge the maximum diameter of the second mouth 120.

Thus, as illustrated, the second closure mechanism 128 is implemented as a drawstring type configuration, such as in a manner analogous to the drawstring implementation of the first closure mechanism 111. For instance, in an example implementation, the second channel 130 is implemented as a casing. In the illustrated example, the second closure line 132 comprises a string that passes through the casing and exits about a slit 138 therein. The second locking mechanism 134 comprises a lock having a string channel 140 that the ends of the second closure line 132 pass through. The second locking mechanism 134 also includes a release 136 that allows the lock to transition between the locked state wherein the second closure line 132 is restricted from sliding through the string channel 140, and the unlocked state wherein the second closure line 132 can slide through the string channel 140.

In the example illustrated in FIG. 1, unlike the first closure mechanism 111, the second channel 130 is on the outside of the container body 102. However, in practice, the second channel 130 can be on the outside or the inside of the container body 102. During a typical procedure, the second mouth 120 will typically be positioned outside the patient's body cavity. As such, the exact positioning of the second locking mechanism 134 is typically less critical than the positioning of the first locking mechanism 116.

In example implementations, the first closure mechanism 111 and the second closure mechanism 128 are completely independent of one another. That is, the first closure mechanism can be operated without having any bearing on the state of the second closure mechanism, and vice-versa. Here, the first closure mechanism 111 and the second closure mechanism 128 are separate structures, each with a different function and purpose during the course of a surgical procedure.

Miscellaneous Example Configurations

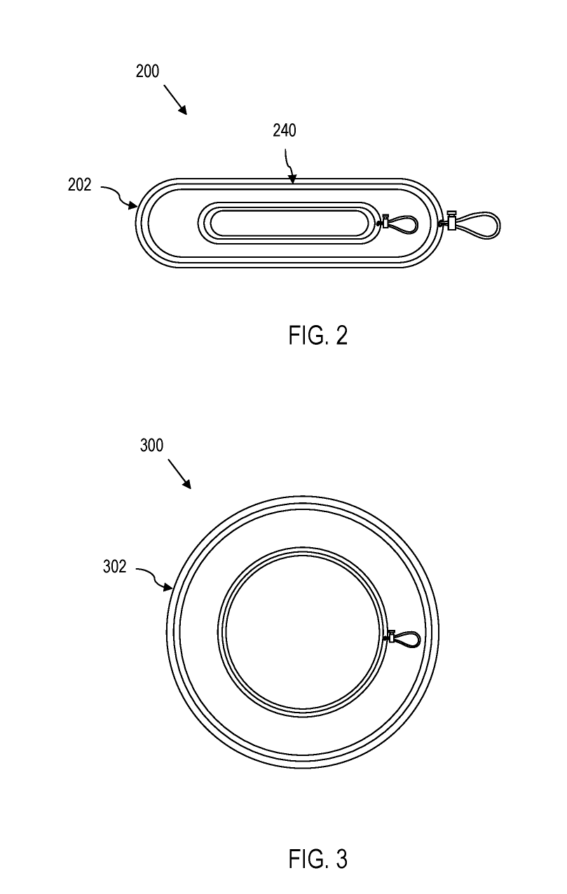

Referring to FIG. 2, a top view of an example surgical isolation drape 200 is illustrated. The surgical isolation drape 200 is analogous to the surgical isolation drape 100 of FIG. 1, and thus can include any combination of the features discussed above. As such, like elements are illustrated with like reference numerals 100 higher. As illustrated, the surgical isolation drape 200 includes a container body 202 that has a generally oval-shaped cross-section. In practice, surgical isolation drape 200 can include any combination of the first closure mechanism, the second closure mechanism, or both. Both closure mechanisms are illustrated solely by way of example. For instance, the first closure mechanism can include a drawstring-type structure as described above. Moreover, the second mouth 120 can include a stent 240 or other structure that assists in keeping the container body 202 open at the second end.

Referring to FIG. 3, a top view of an example surgical isolation drape 300 is illustrated. The surgical isolation drape 300 is analogous to the surgical isolation drape 100 of FIG. 1, and thus can include any combination of the features discussed above. As such, like elements are illustrated with like reference numerals 200 higher. As illustrated, the surgical isolation drape 300 includes a container body 302 that has a generally circular shaped cross-section. The illustrated surgical isolation drape 300 does not include a second closure mechanism to illustrate the flexibility of configurations.

Use of the Surgical Drape in a Medical Procedure

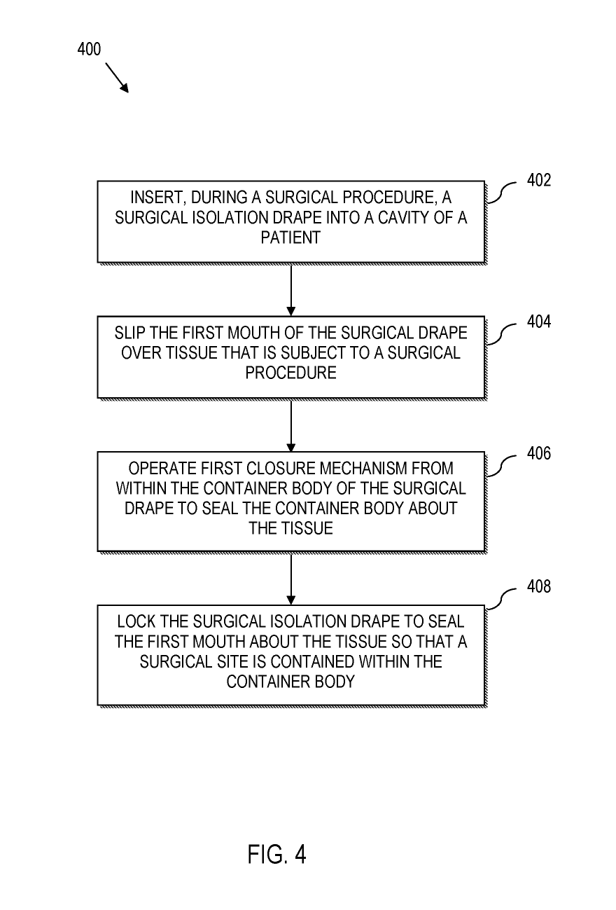

Referring to FIG. 4, a method 400 of using a surgical isolation drape is illustrated. The method 400 comprises inserting at 402, during a surgical procedure such as a laparoscopic procedure, a surgical isolation drape into a cavity of a patient. The surgical isolation drape can include any combination of features as set out and described with reference to FIG. 1-FIG. 3.

For instance, the surgical isolation drape can comprise a medical-grade flexible container body, having an inside surface, a first end that is normally open, thus defining a first mouth, and a second end opposite the first end that is normally open, thus defining a second mouth. In this manner, the container body defines a sleeve when the first mouth and the second mouth are each open.

Also, as described more fully herein, the surgical drape comprises a first closure mechanism. For instance, as described more fully herein, the surgical drape can comprise a first channel inside of the container body proximate the first mouth. A first closure line passes through the first channel, such that access to the first closure line is provided from a position inside the container body. Moreover, a first locking mechanism engages the first closure line, such that access to the first locking mechanism is provided from a position inside the container body. Notably, the first locking mechanism is operable from within the container body. The first locking mechanism is transitioned to a locked state to cause the first closure line to restrict a maximum diameter of the first mouth, and the first closure line can be pulled from within the container body when the first locking mechanism is in an unlocked state to restrict the maximum diameter of the first mouth so as to sealingly close around tissue and create an isolated surgical environment.

The method 400 also comprises slipping at 404, the first mouth of the surgical drape over tissue (e.g., an organ) that is subject to a surgical procedure. For instance, the surgical isolation drape is inserted into the cavity of a patient, leaving the second end outside the patient. The first end is lowered into the surgical location, e.g., a body cavity of the patient, and the first mouth is slipped over tissue that is the subject of the procedure.

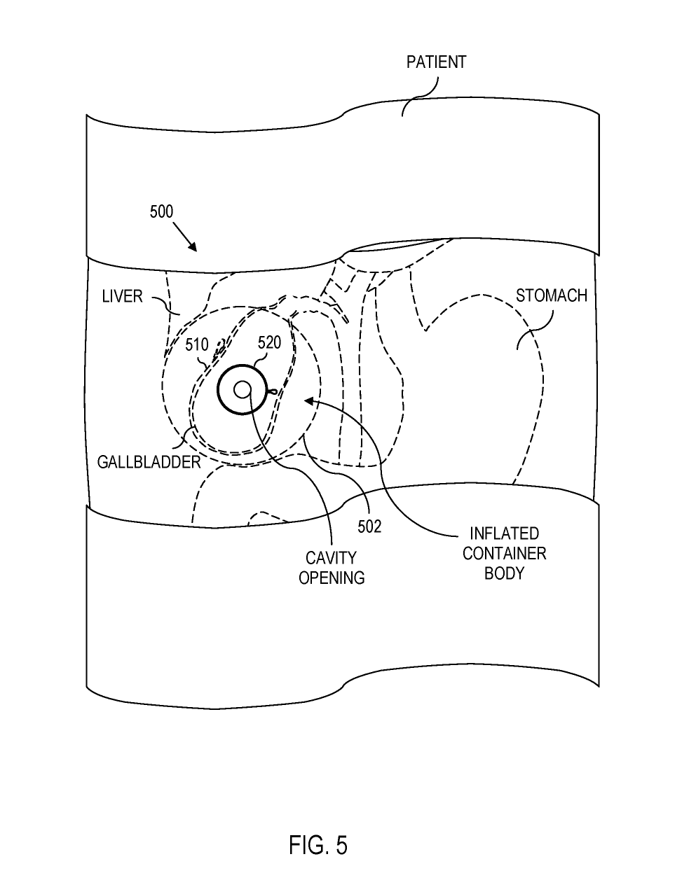

For instance, reference is briefly drawn to an example shown in FIG. 5. Here, an example surgical procedure is schematically illustrated of removing a gallbladder. A surgical drape 500 (e.g., which can include any structures and features described more fully herein with regard to FIG. 1-3), is inserted into a cavity of a patient undergoing the surgical procedure. More particularly, a first mouth 510 of the surgical drape 500 is passed through the cavity of the patient, and is slipped over the tissue of interest, in this instance, a gallbladder. Here, the second mouth 520 can include a stent or other optional structures to assist in holding the surgical drape open outside of the patient body.

Optionally, the surgical drape is inflated to push organs and other tissue out of the way. This also allows room for surgical instruments to be maneuvered more readily. For instance, as best illustrated in FIG. 5, the surgical drape is inflated, pushing the liver and stomach out of the way of the gallbladder, thus increasing room for surgical instruments to be wielded more effectively.

Referring back to FIG. 4, the method 400 also comprises operating the first closure mechanism from within the container body of the surgical drape to seal the container body about the tissue. By way of example, with the drawstring example described more fully herein, the closure mechanism can be operated by pulling, at 406, a first closure line from within the container body of the surgical drape to seal the container body about the tissue (e.g., gallbladder in the example of FIG. 5) that is the subject of the procedure. As such, the the surgical site is contained within the sleeve defined by the container body 102.

Yet further, the method 400 comprises locking, at 408, the first closure mechanism to seal the first mouth about the tissue so that a surgical site is contained within the container body. Keeping with the above example, the first closure mechanism is operated by transitioning the first locking mechanism to its locked state after the first closure line has been pulled to seal the first mouth about the tissue so that a surgical site is contained within the container body.

In certain procedures, the tissue will remain in the patient, such as where a surgical repair is taking place. In other procedures, tissue will be excised. This procedure can be carried out as an iterative process, cutting and tightening the first closure mechanism in turn. Alternatively, such as where a clean excision can be swiftly carried out, the tissue can be excised, then the first mouth can be completely sealed off via the first closure mechanism, thus containing the excised tissue within the container body.

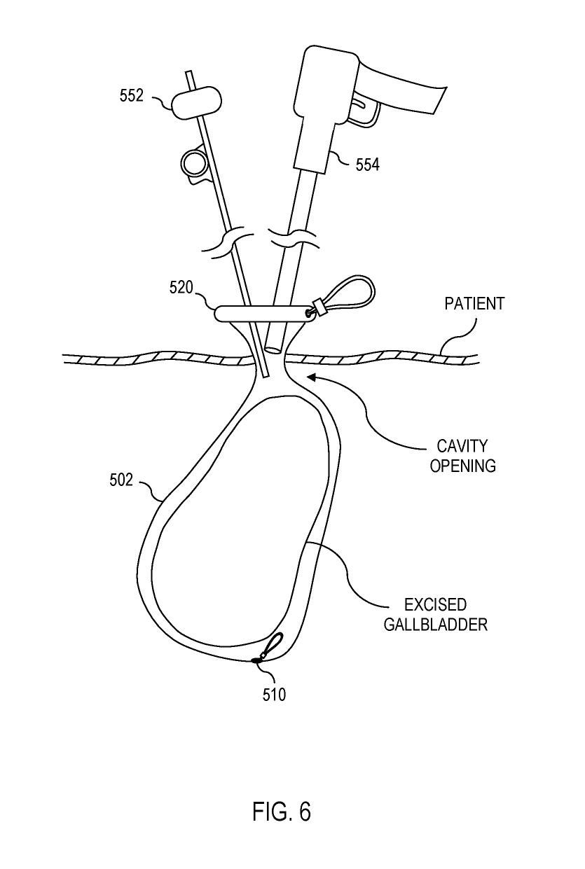

Referring briefly to FIG. 6, after excising the gallbladder, the first mouth 510 of the container body 502 is sealed off, resulting in the surgical drape forming a bag. The second mouth 520 remain open to allow surgical instruments 552, 554 to be passed into the container body 502

Referring generally to FIGS. 4-6, as noted above, in an example implementation, the method 400 can comprise leaving the second mouth of the surgical drape external to the body of the patient undergoing the surgical procedure. Here, the second mouth is left open to allow access to the container body. Using this approach, the method 400 can also include passing medical instruments 552, 554 into the container body such that the medical instruments 552, 554 are not in contact with the cavity of the patient outside the container body 502 of the surgical drape 500. Here, the surgical instruments 552, 554 are arranged according to a standard recognized orientation for the surgical procedure being performed.

Moreover, in an example application, the method 400 includes inserting a morcellation instrument 552 into the container body, optionally closing the second mouth about the morcellation instrument, and performing a morcellation of the tissue contained in the surgical drape. As such, the surgical isolation drape is suitable for procedures including excision and removal of pathology as well as morcellation in a contained environment, such as within an abdominal cavity. Here, the surgical isolation drape avoids the inherent problems of conventional approaches that require the surgeon to excise and manipulate tissues prior to isolation of the tissue from surrounding organs not involved in the primary procedure as well as requiring surgical placement of surgical waste into a bag prior to morcellation as the surgical waste is already be contained using the surgical drape disclosed herein.

In certain example implementations, the method 400 can also include inflating the container body so as to push organs within the body cavity not involved in the procedure out of the surgical field allowing the surgical site to be defined as the area within the container body. Aside from isolating the body cavity from tissue pathology, the inflation of the container body pushes tissue not subject to the procedure (e.g., an organ such as the bowel) away from the immediate operating field after being inflated.

The second opening can remain external to the body and body cavity throughout the procedure. The container body passes through the body cavity wall, thus acts as a conduit to pass surgical devices through without ever contacting the body of the patient undergoing surgery outside of the container body of the surgical drape. The first opening, with the first closure mechanism (e.g., drawstring) closed around the base of the tissue subject to the procedure, isolates the remainder of the body cavity from wastage/spillage products of the surgical tissue during the entire process of the procedure.

In certain procedures, the method can include removing the tissue completely from its original location, closing the first mouth about the tissue as the tissue is removed so that the tissue is set aside within the container body, and completely closing the first mouth, e.g., via the first closure mechanism (e.g., a drawstring) thus totally isolating the tissue from the body cavity.

As another illustrative example, the method 400 can also include closing the first end of the container body trapping the excised tissue within, inserting a tool into the container body through the second mouth, and morcellating tissue in the container body, thus isolating the cavity from surgical debris and waste. Likewise, the method 400 can include inserting surgical instruments through the container body so as to arrange the instruments according to a standard recognized orientation for the surgical procedure being performed.

Miscellaneous

Using the surgical isolation drape according to various aspects of the present disclosure, tissue can be removed from its original location and can be set aside within the container body of the surgical isolation drape. The first (internal) opening can be completely closed via its closure mechanism, (e.g., drawstring sealing closed a first mouth defining the internal opening) thus totally isolating the abdomen or other patient cavity from the surgical tissue/debri and waste.

Moreover, the surgical isolation drape can be used in procedures requiring the partial or complete removal of tissue, cysts, tumors, or other natural or unnatural bodily features.

The container body itself is made of a clear material with some degree of elasticity (e.g., plastic). The clear material allows for visualization of anatomy beyond the container body and pathology therefore improving use of the surgical isolation drape. Moreover, the surgical isolation drape is preferably elastic. The elastic characteristics enable trocars and surgical instruments to pierce the container body. However, the container body continues to be able to maintain an adequate seal around the trocars and thus continue to isolate the body cavity. Accordingly, surgical instruments can be arranged in a standard recognized orientation for a given surgery.

Still further, excised tissue may comprise a mixture of soft tissue, fibroids, muscle, and other firm tissue, fluid-filled tissue such as from cysts, etc. The container body is sufficiently flexible to contain and prevent spillage, leakage and other escape of materials captured into the container body. Thus, cancerous tissue cannot shed cancerous cells back into the body of the patient as a result of the procedure.

Throughout the specification, numerous example embodiments are described by way of illustration and not by way of limitation. In this manner, various embodiments are described that draw particular reference to a particular feature or group of features for sake of simplified discussion and explanation. In practice, features from multiple embodiments described herein can be combined in various configurations.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure.

Having thus described the disclosure of the present application in detail and by reference to embodiments thereof, it will be apparent that modifications and variations are possible without departing from the scope of the disclosure defined in the appended claims.

What is claimed is:

1. A surgical isolation drape comprising:
a medical-grade flexible container body, the flexible container body having:
an inside surface;
a first end that is normally open, thus defining a first mouth; and
a second end opposite the first end that is normally open, thus defining a second mouth;
wherein:
the container body defines a sleeve when the first mouth and the second mouth are each open; and
a first closure mechanism attached to the container body that is operable from a position inside the container body to open and close the first mouth, wherein the first closure mechanism is operated to restrict the maximum diameter of the first mouth so as to sealingly close around a tissue of interest and create an isolated surgical environment.

2. The surgical isolation drape of claim 1 further comprising:
a first channel inside of the container body proximate the first mouth; and
a first locking mechanism that engages the first closure mechanism, where access to the first locking mechanism is provided from a position inside the container body;
wherein:
the first closure mechanism comprises a first closure line;
the first locking mechanism is operable from within the container body;
the first locking mechanism is transitioned to a locked state to cause the first closure mechanism to restrict a maximum diameter of the first mouth; and
the first closure mechanism is operated from within the container body when the first locking mechanism is in an unlocked state to restrict the maximum diameter of the first mouth.

3. The surgical isolation drape of claim 2, wherein:
the first channel comprises an internal casing positioned on the inside surface of the container body;
the first closure line comprises a string that passes through the internal casing and exits the container body about a slit therein so as to exit the casing inside the container body; and
the first locking mechanism comprises:
a lock having a string channel that the string passes through; and
a release that allows the locking mechanism to transition between the locked state wherein the string is restricted from sliding through the string channel, and the unlocked state wherein the string can slide through the channel.

4. The surgical isolation drape of claim 2 further comprising:
a second channel proximate the second mouth;
a second closure line passing through the second channel; and
a second locking mechanism that engages the second closure line;
wherein:
the second locking mechanism is transitioned to a locked state to cause the second closure line to restrict a maximum diameter of the second mouth; and
the second closure line can be pulled when the second locking mechanism is in an unlocked state to restrict the maximum diameter of the second mouth.

5. The surgical isolation drape of claim 4, wherein:
the second channel is on the outside of the container body; and
the second closure line is separate from the first closure line such that operation of the first closure line is independent of operation of the second closure line.

6. The surgical isolation drape of claim 4, wherein:
the second channel comprises a casing;
the second closure line comprises a string that passes through the casing and exits about a slit therein; and
the second locking mechanism comprises:
a lock having a string channel that the string passes through; and
a release that allows the lock to transition between the locked state wherein the string is restricted from sliding through the string channel, and the unlocked state wherein the string can slide through the channel.

7. The surgical isolation drape of claim 6, wherein:
the casing of the second channel is an external casing formed with an outside surface of the container body.

8. The surgical isolation drape of claim 1, wherein:
the first closure mechanism enables the first mouth to be drawn into such sealing relationship with the tissue of interest during a surgical procedure to allow isolation of a body cavity and surrounding tissues from the tissue of interest during the surgical procedure.

9. The surgical isolation drape of claim 1, wherein:
the container body is a clear, elastic material; and
the isolation drape forms a vessel configured to be fillable with a gas defining an inflated drape that expands a body cavity allowing for visualization and access to various tissues and organs of the body cavity.

10. The surgical isolation drape of claim 1, wherein:
the first closure mechanism enables the first mouth to be closed off, either entirely or against tissue to define a closed end of the container body.

11. The surgical isolation drape of claim 1, wherein:
the container body is sufficiently elastic to allow surgical instruments to be inserted within the container body without compromising the isolation of the body cavity while the surgical instruments are arranged in a standard recognized orientation for a given surgery.

12. A method of using a surgical isolation drape, comprising:
inserting, during a surgical procedure, a surgical isolation drape into a cavity of a patient, the surgical isolation drape comprising:
a medical-grade flexible container body, the flexible container body having:
an inside surface;
a first end that is normally open, thus defining a first mouth; and
a second end opposite the first end that is normally open, thus defining a second mouth;
wherein:
the container body defines a sleeve when the first mouth and the second mouth are each open; and
a first closure mechanism attached to the container body that is operable from a position inside the container body to open and close the first mouth, wherein the first closure mechanism is operated to restrict the maximum diameter of the first mouth so as to sealingly close around a tissue of interest and create an isolated surgical environment;
slipping the first mouth of the surgical drape over the tissue of interest that is subject to a surgical procedure;
operating the first closure mechanism from within the container body of the surgical drape to seal the container body about the tissue; and
locking the first closure mechanism to seal the first mouth about the tissue so that a surgical site is contained within the container body.

13. The method of claim 12 further comprising:
leaving the second mouth external to the body of the patient undergoing the surgical procedure; and
passing surgical instruments into the container body such that the surgical instruments are not in contact with the cavity of the patient outside the container body, wherein the surgical instruments are arranged according to a standard recognized orientation for the surgical procedure being performed.

14. The method of claim 12 further comprising:
inserting a morcellation instrument into the container body;
closing the second mouth about the morcellation instrument; and
performing a morcellation of the tissue contained in the surgical drape.

15. The method of claim 12 further comprising:
inflating the container body so as to push organs within the body cavity of the patient away from the surgical field allowing the surgical site to be more well defined as the area within the container body.

16. The method of claim 12 further comprising:
removing the tissue completely from its original location;
closing the first mouth about the tissue as the tissue is removed so that the tissue is set aside within the bag; and
completely closing the first mouth via the first closure mechanism thus totally isolating the tissue from the body cavity.

17. The method of claim 12 further comprising:
closing the first end of the container body;
inserting a tool into the container body through the second mouth; and
morcellating tissue in the container body, thus isolating the cavity from surgical debris and waste.

18. A surgical isolation drape comprising:
a medical-grade flexible container body, the flexible container body having:
an inside surface;
a first end that is normally open, thus defining a first mouth; and
a second end opposite the first end that is normally open, thus defining a second mouth;
wherein:
the container body defines a sleeve when the first mouth and the second mouth are each open;
a first channel inside of the container body proximate the first mouth;
a first closure line that passes through the first channel, such that access to the first closure line is provided from a position inside the container body; and
a first locking mechanism that engages the first closure line, where access to the first locking mechanism is provided from a position inside the container body;
wherein:
the first locking mechanism is operable from within the container body;
the first locking mechanism is transitioned to a locked state to cause the first closure line to restrict a maximum diameter of the first mouth; and
the first closure line can be pulled from within the container body when the first locking mechanism is in an unlocked state to restrict the maximum diameter of the first mouth so as to sealingly close around tissue and create an isolated surgical environment.

19. The surgical isolation drape of claim 18 further comprising:
a second channel proximate the second mouth;
a second closure line passing through the second channel; and
a second locking mechanism that engages the second closure line;
wherein:
the first channel comprises an internal casing positioned on the inside surface of the container body;
the first closure line comprises a string that passes through the internal casing and exits the container body about a slit therein so as to exit the casing inside the container body;
the first locking mechanism comprises:
a lock having a string channel that the string passes through; and
a release that allows the locking mechanism to transition between the locked state wherein the string is restricted from sliding through the string channel, and the unlocked state wherein the string can slide through the channel;
the second channel is on the outside of the container body;
the second channel comprises a casing;
the second closure line comprises a string that passes through the casing and exits about a slit therein;
the second locking mechanism comprises:
a lock having a string channel that the string passes through; and
a release that allows the lock to transition between the locked state wherein the string is restricted from sliding through the string channel, and
the unlocked state wherein the string can slide through the channel;
the second locking mechanism is transitioned to a locked state to cause the second closure line to restrict a maximum diameter of the second mouth; and
the second closure line can be pulled when the second locking mechanism is in an unlocked state to restrict the maximum diameter of the second mouth.

20. The surgical isolation drape of claim 18, wherein:
the second closure line is separate from the first closure line such that operation of the first closure line is independent of operation of the second closure line.
